(19) United States
(12) Patent Application Publication
(10) Pub. No.: US 2010/0124584 A1
(43) Pub. Date: May 20, 2010

(54) APPARATUS AND METHOD FOR CONTINUOUS FERMENTATION

(76) Inventors: Benjamin Alexander, Portland, ME (US); Garth Anton Cambray, Grahamstown (ZA); Eli Cayer, Portland, ME (US)

Correspondence Address: CHRIS A. CASEIRO, VERRILL DANA, LLP, ONE PORTLAND SQUARE, PORTLAND, ME, 04112-0586, US

(21) Appl. No.: 12/274,447

(22) Filed: Nov. 20, 2008

(51) Int. Cl. C12G 3/02; C12P 7/06

(52) U.S. Cl. 426/11; 99/276

(57) ABSTRACT

A hybrid continuous fermentation system and related process for fermenting sugars to produce alcohol containing liquors. The system includes a fermentor arranged with a packed bed region and a fluidized bed region. A yeast bed is retained in the packed bed region using a retention matrix and flocculation. A sugar supply is directed through the yeast bed with sufficient dwell time for satisfactory sugar conversion. The fluidized bed region is arranged to allow fermented product to pass through the fermentor while allowing yeast cell growth and retention. A nutrient may be added to the fermentor to aid in yeast flocculation. The fermentor is sized and shaped to allow sufficient dwell time of the sugar supply in the packed bed region and in the fluidized bed region to maximize sugar conversion to ethanol by fermentation.

### BACKGROUND OF THE INVENTION

1. Field of the Invention

The present invention relates to the production of ethanol by fermentation including, but not limited to, alcoholic beverages such as mead. More particularly, the present invention relates to devices and methods to produce such alcoholic liquors in a continuous process. The present invention is a continuous fermentation reactor apparatus and the method for making alcoholic liquors (liquors being mixed solutions containing alcohol) using such an apparatus.

2. Description of the Prior Art

In general, the production of ethanol-hereafter referred to as alcohol involves combining a source of sugar with a catalyst capable of oxidizing the sugar to break it down into ethanol and byproducts, such as carbon dioxide. For wine, the sugar source is grapes; for mead (a fermented honey beverage), the sugar source is honey or honey and other sources (e.g., fruit juice). These sources are used to produce, by fermentation, the ethanol alcoholic beverages. The sources may be referred to by several terms, but for the purpose of describing the present invention, they will be referred to as must. It is to be understood that the term “must” as used herein describes the source of a sugar and is not intended to limit the invention to a specific type of source. The ethanol forms the alcohol basis of the beverage, with the must and other selectable additives providing the flavor of the beverage. Yeast is the catalyst of choice used to facilitate the conversion of sugars to alcohol.

The production of alcoholic liquors occurs in two principal stages, aerobic (growth) and anaerobic fermentation. In the aerobic stage, a catalyst and must are introduced to a reaction vessel and combined in the presence of oxygen. During the anaerobic phase, the presence of oxygen is conducive to catalyst/yeast growth which is important for the rapid fermentation of the remaining sugars into ethanol and carbon dioxide. The anaerobic phase occurs without oxygen whereby the remaining sugars are converted, by fermentation, to ethanol and carbon dioxide by yeasts. Fermentation of sugar-containing musts may be carried out either in a batch process or a continuous process. An effective process involves efficient conversion of the starter materials to sugar and then the sugar to ethanol. The theoretical maximum yield for ethanol conversion is 0.51 g ethanol per gram of glucose in the must.

As those in the art realize, batch fermentation involves the production of a finite quantity of alcohol in a reaction vessel. The aerobic and anaerobic stages occur in the same reactor. Formula components in selected ratios are combined in the reactor, oxygen is then introduced for a period of time at which point the reactor is closed and fermentation of the remaining sugars occurs. Upon completion of the fermentation process, the reactor is opened and the contents removed. The yeast may be reused a few times in batch reactions, but with each reaction, risk of contamination by non-desirable microorganisms increases and the yeast can become stressed in each subsequent use, resulting in reduced efficiency. Furthermore, in a batch process, some of the sugar in the must is consumed resulting in yeast growth, producing biomass instead of alcohol, resulting in a fermentation well below the theoretical yield. The batch process is effective in that the fermentation process can be well controlled and the product quality is consistent. However, the batch process is not particularly efficient and, therefore, has commercial limitations. Moreover, for small-scale producers, relatively large volumes of alcohol production can be difficult to achieve without substantial capital investment.

With respect to the production of mead in particular, the mixture of sugars glucose, fructose and sucrose in honey must require the existence of yeast in different metabolic states for effective conversion to occur. In a batch reactor, or any reactor in which the yeast state is uniform, honey must conversion is slowed by the switching of metabolic states required to metabolize different sugars. These switches in metabolic state, required to process different sugars, make batch mead making a slow process.

Continuous fermentation processes can address many of the limitations associated with the batch fermentation process. These include higher conversion rates, faster fermentation rates, improved product consistency, reduced product losses and environmental advantages. (See Biotechnology Letters, Vol. 28, pp. 1515-1525, 2006, incorporated herein by reference.) These advantages benefit the small-scale producer in particular, to enable the production of substantial quantities without the costly investment associated with ethanol producing plants. In general, the continuous process includes the use of one or more passes through reaction vessels wherein yeast of the feed must is recycled while the fermentation process occurs in the vessel. Starter components are transferred into the vessel and the alcoholic liquor drawn from that vessel without halting the fermentation process. A number of continuous fermentation systems have been described. In general, however, continuous fermentation processes have limitations associated with increased costs and/or falling short of the maximum ethanol conversion desired in alcohol production.

One example of a known continuous fermentation process involves the use of multiple reaction vessels, with aerobic fermentation and anaerobic fermentation occurring in different vessels. The vessels may be arranged in a cascading series. Such an arrangement can be difficult to maintain and is of limited efficiency, with drop off in reactivity likely occurring at the transfer from one vessel to another. In another example of a continuous fermentation process, a single reaction vessel is employed. The process substantially immobilizes the yeast in a substantially anaerobic state. As a result, yeast growth cannot occur at a rate necessary to maintain sugar conversion unless the must input rate is very slow. It is therefore necessary to add fresh yeast regularly to the vessel to keep must introduction at a reasonably effective rate. The need to add fresh yeast is time consuming and inhibits the viability of the yeast (and thus the efficiency of the fermentation) because the yeast must be reintroduced to the fermentor in a different metabolic state.

Yet another type of continuous fermentation reactor involving the use of a single reactor seeks to improve ethanol conversion by directing gas such as carbon dioxide or oxygen into the bottom of the vessel creating “airlift,” which is intended to maintain a well mixed state within the reactor. This has the effect of increasing mass transfer and, therefore, fermentative capacity. Unfortunately, such a fluidized bed configuration results in a loss of yeast/catalyst from the reactor and thus operates at a decreasing productivity. That is, the airlift fermentation process is suitable to sustain yeast growth and sugar conversion; however, will inevitably result in yeast loss and therefore reduced productivity.

Yeast immobilization has been studied substantially to mitigate the limitations associated with yeast recycling. Common immobilization techniques include: 1) attachment to a surface; 2) entrapment within a porous matrix; 3) containment behind a barrier; and 4) self adhesion, also referred to as flocculation. Attachment to a surface is achieved by allowing yeast cells to adhere to other organic and inorganic substances and may be induced artificially or occur naturally, the latter being preferred in beverage/food production. Entrapment within a porous matrix requires the introduction of a relatively inert material with interstices of sufficient size to capture and retain the yeast particulate. Containment behind a barrier is a similar mechanism in that the barrier material should be relatively inert and its pores small enough to retain the yeast. Flocculation or self adhesion is a mechanism by which yeast (and other organisms) bond together in response to stress. Flocculation is commonly relied upon in the brewing industry to separate yeast from fermented wort. Research has demonstrated that flocculation can be an effective immobilization tool; however, maintaining control of this mechanism can be difficult because of the numerous parameters that affect it including, for example, nutrient conditions, agitation, Calcium concentrations, pH, fermentation temperature, yeast handling, and storage conditions (See Biotechnology Letters, Vol. 28, pp. 1515-1525, 2006.).

Additional reactor configurations include packed bed reactors, fluidized bed reactors; gas lift reactors (or stirred reactors) and membrane cell recycle reactors. These systems include three phases: solid (carrier matrix and biomasss); liquid (must); and air, oxygen or other gas feed. In designing any system, simplicity is desirable. For that reason, most commercially viable fermentor configurations comprise either packed bed or fluidized bed systems, due to the lack of moving parts in the fermentor, and small plant operation requirements.

In general, it would be desirable to have a fermentation process and associated system to produce liquor as close to the theoretical maximum conversion rate as possible. Further, it would be desirable to have a continuous fermentation process that provides for efficient yeast conversion with minimal need for new yeast introduction. Yet further, it would be desirable to provide such a continuous fermentation process and system embodied in a single reaction vessel that avoids the costs associated with cascading reactors or airlifting through the reaction vessel.

### SUMMARY OF THE INVENTION

It is an object of the present invention to provide a fermentation process and associated system to produce alcohol liquors as close to the theoretical maximum conversion rate of sugar to ethanol as possible. It is also an object of the present invention to provide such a process and system that achieves such a conversion rate without the limitations of a batch reaction process. Further, it is an object of the present invention to provide a continuous fermentation process that provides for efficient yeast conversion without the need for new yeast introduction. Yet further, it is an object of the present invention to provide such a continuous fermentation process and system embodied in a single reaction vessel which utilizes yeast immobilization. Rather than airlifting to increase mass transfer or yeast growth (and thus fermentative capacity) through the reaction vessel.

These and other objects are achieved with the present invention, which is a single reaction vessel continuous fermentation system and related process. The system is arranged to include a hybrid bed comprising a packed bed and a fluidized bed in the same reaction column. The system is applicable to the fermentation of mixed fermentable sugar solutions, such as those found in honey, but not limited thereto. For example, the fermentation of enzyme-treated sugar beet juice, apple, pineapple, tangello, prickly pear and even straight sugar solution and molasses, but is not limited thereto. However, with respect to the production of mead, this hybrid arrangement creates an environment in which the yeast is in different metabolic states through the column. As a result, the glucose, fructose and sucrose of the honey is consumed more efficiently than other systems with the yeast metabolizing first glucose in the bottom of the fermentor, then fructose, and finally sucrose and other sugars as the must is pumped up through the column. This creates a stratification of yeasts that are in different metabolic states in the reactor.

The packed bed portion of the reaction column includes yeast immobilization arrangements where little cell growth occurs so that the yeast may generate sugar conversion rates approaching theoretical maximum. The invention also includes the use of a flocculent yeast strain that further enhances conversion rates by causing the yeast to clump in a way that immobilizes it. The fluidized bed portion of the system is located above the packed bed portion in the column. The transition from the packed bed to the fluidized bed is gradual and fluidization is created by the carbon dioxide produced in the sugar conversion phase occurring in the packed bed.

The yeast immobilization arrangement of the present invention is sufficient to keep cell growth minimal, porous enough to allow generated gases to rise to the top of the column, and retains the yeast so that little live yeast passes out of the reactor with the generated gases and outflow of liquor. Further, as noted, yeast forced to the fluidized bed portion aided by its flocculent properties and immobilization, is able to fall back to the packed bed portion of the vessel. The yeast then performs its sugar conversion function in the packed bed. It is therefore not necessary to replenish the yeast supply to maintain the reaction process as close to maximum conversion at all times.

As a result of the anaerobic conditions within the reactor, the yeast is considered to be in a stationary phase whereby little or no cell growth occurs. This has the long-term effect of maintaining a continuous culture of yeast that evolves, adapting to the environment. The limited cell growth replaces older weaker cells which eventually flow out of the reactor.

The hybrid continuous fermentation reactor apparatus and associated process of the present invention yield high sugar conversion rates to make alcohol liquors. This and other advantages of the present invention will become apparent upon review of the following detailed description, the accompanying drawings and the appended claims.

### BRIEF DESCRIPTION OF THE DRAWINGS

FIG. 1 is a side view of the hybrid continuous fermentation reactor of the present invention.

FIG. 2 is s simplified flow diagram of the primary steps of the fermentation process of the present invention.

### DETAILED DESCRIPTION OF THE PREFERRED EMBODIMENTS

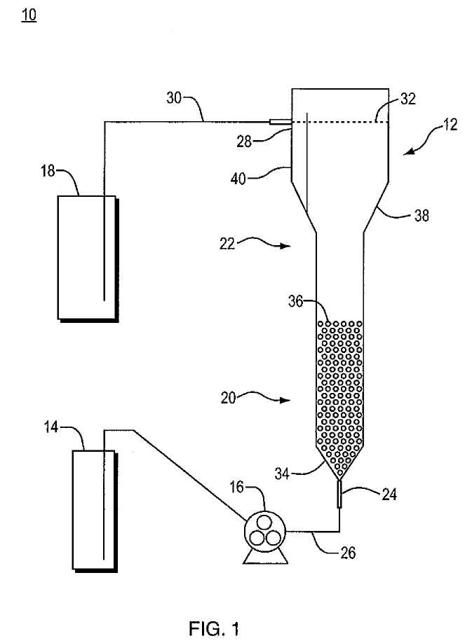

A hybrid continuous fermentation reactor system 10 is shown in FIG. 1. The system 10 includes a fermentor 12, a sugar source 14, a sugar source delivery device 16 and an alcohol liquor collector 18. The system 10 may be used to ferment a sugar supply in a continuous process to produce an alcoholic liquor such as, but not limited to, wine, beer or mead. The system 10 substantially completely ferments the sugar supply to produce an alcoholic beverage that is substantially void of the sugar supplied. This is achieved through higher conversion rates, faster fermentation rates, improved product consistency and reduced product loss.

The fermentor 12 includes a packed bed region 20 and a fluidized bed region 22. The packed bed region 20 includes a yeast bed and is arranged to enable anaerobic fermentation of the sugar supply to produce ethanol. The fluidized bed region 22 is arranged to enable yeast that flocculates to return to the packed bed region 20. The packed bed region 20 includes a retention matrix to facilitate yeast immobilization therein to establish the yeast bed in a form sufficient to maximize sugar breakdown but without binding the yeast so much that yeast cannot pass into the fluidized bed region 22. Yeast growth occurs slowly across the whole column. The retention matrix may be, but is not limited to being, an organic material, such as herbal tea particles or ginger root cubes.

The fermentor 12 includes an inlet port 24 at the entry to the packed bed region 20. The inlet port 24 is coupled to fermentor inlet conduit 26 and arranged to transport the must and any desired additives from the sugar source 14 via the delivery device 16. The delivery device 16 may be a peristaltic pump, for example. The delivery device 16 is operable to direct the sugar supply to the fermentor 16 at a selectable flow rate. The fermentor 12 also includes an outlet port 28 at the exit of the fluidized bed region 22. The outlet port 28 is coupled to fermentor outlet conduit 30 and arranged to transport distillate form the fermentor 12 to the liquor collector 18. The outlet port 28 is positioned on the fermentor 12 at a location that establishes a fluid height line 32 that is the top of the fluid column in the fermentor 12. Fermented fluid and fermenting off gases, such as carbon dioxide, pass out of the fermentor 12 through the outlet port 28

An aspect of the fermentor 12 that enhances the fermentation process carried out with the present invention is its shape. Specifically, the fermentor 12 is a column with four primary sections. Inlet section 34 includes inlet port 24 and a portion of the packed bed region 20. The inlet section 34 is arranged to provide facilitate the entry of must into the yeast bed. The inlet section 34 may be conical in shape. Primary matrix retention section 36 is arranged to provide sufficient dwell time of active yeast in the presence of the retention matrix, the sugar source, and any other additives of interest including, but not limited to, any nutrients and pH buffers considered useful in maximizing yeast effectiveness. The retention matrix is also capable of adherence of yeast to its surface forming a biofilm that further enhances immobilization. The primary matrix retention section 36 is positioned above the inlet section 34 when the fermentor 12 is operating. The primary matrix retention section 36 may be cylindrical in shape and contains the remainder of the packed bed region 20 of the fermentor 12. It may also include a portion of the fluidized bed region 22.

With continuing reference to FIG. 1, the fermentor 12 also includes a flow rate reduction section 38 positioned above the primary matrix retention section 36 when the fermentor 12 is operating. The flow rate reduction section 38 includes the fluidized bed region 22 of the fermentor 12 and is arranged to slow the rate of fluid movement upwardly through the height of the fermentor 12. Yeast that is relatively heavier than liquid and gas in the fluid but entrained in the fluid slows in the flow rate reduction section 38 and is forced to the perimeter of the fluid column within the fermentor 12. When the yeast slows and reaches the interior walls of the fermentor 12 at the flow rate reduction section 38, its descent is gradual and does not disrupt upward fluid movement at the interior of the fermentor 12. While in the anaerobic, largely sugar and nutrient depleted, environment of the fluidized bed region 22 including the flow rate reduction section 38, yeast cells grown drop back into the primary matrix retention section 36 for use in the break down of new sugars entering the fermentor 12 thus retaining biomass and enhancing fermentative efficiency and productivity. The flow rate reduction section 38 may be conical in shape and in the present embodiment of the invention has dimensions exceeding the dimensions of the primary matrix retention section 36.

The fermentor 12 includes a final yeast separation section 40 positioned above the flow rate reduction section 3 8 when the fermentor 12 is operating. The final yeast separation section 40 includes the remainder of the fluidized bed region 22 of the fermentor 12. The final yeast separation section 40 is arranged to further slow fluid flow rate within the fermentor 12 so that any remaining yeast or any other particulates that may be entrained in the fluid are directed to the interior perimeter of the fermentor 12 to fall back down to the packed bed region 20. The fluid is at peak alcohol content at the fluid height line 32, where it and fermentation gases exit the fermentor 12. The dimensions of the fermentor 12 at the final distillation section 40 are greater than the dimensions of the fermentor 12 at the flow rate reduction section 38. The final yeast separation section 40 may be cylindrical in shape.

The identified sections of the fermentor 12 are sized to maximize break down of the sugar supply flowing therein. The specific dimensions of the fermentor 12 may be defined based on the particular alcoholic liquors to be produced and the yeast and other additives used in that process. In general, the column height and cross sectional dimensions of the fermentor 12 at these sections, and the rate of flow of sugar supply into the fermentor 12, are selected to ensure sufficient dwell time of the sugar in the fixed bed region 20, and sufficient yeast dwell time in the fluidized bed region 22 to achieve optimal conversion of sugar to alcohol. The transition section that is the flow rate reduction section 38, is designed to maximize removal of fluidized yeast from the fluid column and its return back to the yeast bed. This arrangement ensures continuous flow of yeast within the fermentor 12 where desired, with the fermentation gas carbon dioxide pushing freely suspended yeast up the middle of the fluid column until that flow slows and the yeast drops back down to the yeast bed along the inside wall of the column.

One example of the design of the fermentor 12 found to be suitable for this purpose has the inlet section ÷60 millimeters (mm) in length, the primary matrix retention section 36 910 mm in length, the flow rate reduction section 38 80 mm in length, and the final distillation section 40 80 mm in length. The primary matrix retention section 36 is 50 mm in diameter and the final distillation section 40 190 mm in diameter. The inlet section 34 is arranged with a conical angle of reduction of about 30° and the flow rate reduction section 38 is arranged with a conical angle of reduction of about 30°. The rate of flow of the sugar supply into the fermentor 12 for this particular design of the fermentor is in the range of about one milliliter (ml) per minute to about four ml per minute. It is to be understood that the present invention is not limited to such specific features and is scalable to various larger production volumes.

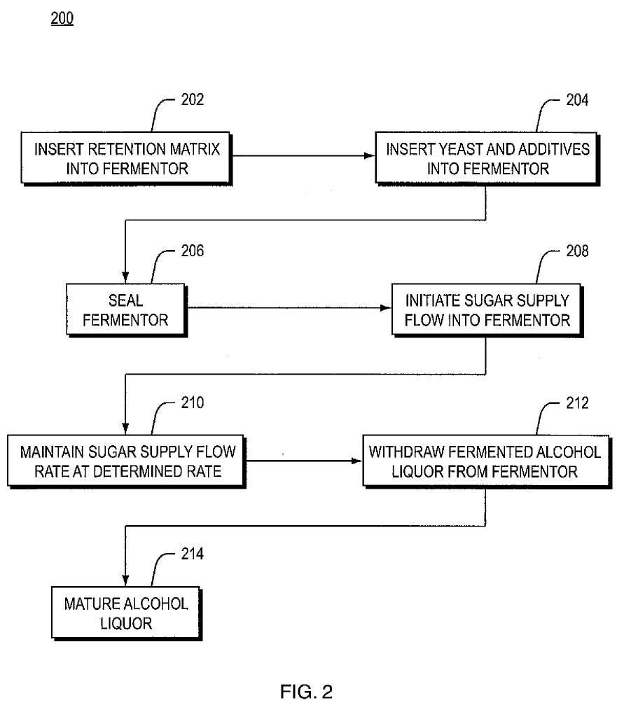

A process 200 of fermentation that is part of the present invention is described with respect to the representation flow diagram of FIG. 2, which illustrates primary steps of the process 200. Before proceeding with the fermentation process, the desired final alcoholic liquor product is selected. That information is used to select a sugar supply and associated additives of interest. Further, the fermentation yeast, additives and retention matrix are selected and the fermentor 12 is sized and dimensioned. The selected sugar supply is contained in sugar source 14, which may be a sugar supply contained in a bottle, bag, or other form of container.

One additive considered useful in the fermentation of a honey must is a yeast nutrient identified as ideal mead making water emulator available from Maine Mead Works of Portland, Me. This product adds the salts to any potable drinking water which meets the requirements of the US Safe Drinking Water Act of 1974, and subsequent amendments, and will convert that water to an ideal mead making water. The nutrient is used to treat the honey must so as to provide anions and cations which enhance yeast flocculation. That is, the nutrient facilitates yeast cell clumping that ensures yeast will adhere to the matrix and/or fall back from the fluidized bed region 22 to the packed bed region 20. That flocculation and the retention matrix together aid yeast immobilization, which as indicated, in turn, improves sugar conversion efficiency.

Upon completion of these initial steps, which may be carried out by the producer of the final product or by a supplier to the producer of the final product, the fermentation process 200 may proceed.

A first primary step of the process 200 involves opening the fermentor 12, which has been sterilized prior to this by soaking for 24 hours in a 1 molar sodium hydroxide solution, followed by rinsing with water which has been boiled and cooled to 80° C., and inserting the retention matrix into the inlet section 34 and the primary matrix retention section 36, step 202. As earlier noted, the retention matrix may be herbal tea particles or ginger root, for example. The next primary step of the process 200 includes inserting into the fermentor 12 the yeast starter and any other fermentation additives of interest, step 204. Steps 202 and 204 may be performed simultaneously. Next, the fermentor 12 is temporarily or permanently sealed, step 206. The delivery device 16 is set or fixed at the calculated fluid flow rate and activated to cause the sugar supply to be drawn from the sugar source 14 and forwarded into the fermentor 12, step 208. The flow rate selected is maintained and adjusted as needed so as to ensure that the yeast remains in a semi-stationary phase in the packed bed region 20 so that yeast reproduction there is minimal. The flow rate is also selected to ensure that the yeast in the packed bed region 20 is fed the optimal amount of sugar to enable complete fermentation while also allowing for the continually evolving yeast culture to develop inside the fermentor 12, whereby stronger yeast cells develop and replace weaker yeast cells, which weaker cells remain entrained in the fluid column and eventually pass out of the fermentor 12.

In order to ensure an effective continuous fermentation process, the delivery device 16 should be operated so that the flow rate of the sugar supply is maintained substantially at a constant rate, step 210. The fluid in the fermentor 12 is permitted to dwell for a time defined by the flow rate and fermentor dimensions. When the fermentation process has been completed for fluid located at the top of the fermentor 12, the alcohol liquor made is permitted to exit the fermentor 12, step 212. There may be a screen or other form of filtering arrangement at the fluid height 32 or the outlet port 28 to limit the passage of spent yeast cells or other particulate to the fermented liquor collector 18. The alcohol liquor is directed to the collector 18 for maturation for a selectable period of time, step 214. The maturation time may be determined by the producer; however, the efficiency of production provided by the present invention results in a product that requires significantly less maturation time than prior batch processes. Mead for example, often requires one to two years of maturation. With the present invention, mead may be produced and ready for market in four to six weeks.

The hybrid continuous fermentation reactor system 10 and the continuous fermentation process 200 of the present invention combines the attributes of the packed-bed and fluidized-bed continuous fermentation processes. The resultant high biomass retention rates obtained yield high ethanol output per weight of fermented sugar consumed, including approaching the theoretical maximum yield of 0.51 gram of ethanol per gram of sugar consumed. Mead produced using the present invention requires only 97.5 g of honey sugars (containing honey and water) to generate an ethanol content of 12.3% by volume. On the other hand, 97.5 g of honey sugars typically only yields an ethanol content of 10% by volume for the same quantity of honey sugars.

As indicated, the hybrid continuous fermentation reactor system 10 and the related continuous fermentation process 200 of the present invention has been used to convert a mixed sugar solution (such as honey and water or honey, apple juice and water) to a beverage with more than 12% alcohol by volume. The must used to make that beverage measured 21.32 Degrees Brix prior to passing through the fermentor 12, with the resultant liquor exiting the fermentor 12 void of any residual sugar. The anaerobic environment within the fermentor 12 resulted in a conversion rate near the theoretical maximum and an efficient clean fermentation that was void of any volatile compounds, thus reducing the required maturation period.

The present invention has been described with respect to one or more example embodiments. Nevertheless, it is to be understood that various modifications may be made without departing from the spirit and scope of the invention as described by the following claims. Further, the steps of the process described herein may occur in parallel or in different order without deviating from the spirit and scope of the invention as described by the following claims.

What is claimed is:

1. A continuous fermentation reactor system comprising:
a. a sugar source including a sugar supply;
b. a delivery device coupled to the sugar source;
c. a fermentor coupled to the delivery device, wherein the fermentor is arranged to receive the sugar supply from the delivery device, wherein the fermentor is a single fermentor including a packed bed region and a fluidized bed region, and wherein the fermentor includes an outlet port; and
d. a fermented alcohol liquor collector coupled to the fermentor to receive fermented fluid from the outlet port.

2. The system of claim 1 further comprising a yeast bed located in the packed bed region, wherein the delivery device is arranged to direct the sugar supply to the yeast bed in the packed bed region.

3. The system of claim 2 further comprising a retention matrix in the packed bed region, wherein the retention matrix is selected to aid in immobilizing the yeast bed.

4. The system of claim 3 wherein the retention matrix is selected from commonly available food grade items such as ginger root and herbal tea particles.

5. The system of claim 2 further comprising a yeast nutrient added to the must to aid flocculation in the packed bed region.

6. The system of claim 1 wherein the fermentor includes a primary matrix retention section and a final retention section.

7. The system of claim 6 wherein the fermentor includes a flow rate reduction section between the primary matrix retention section and the final yeast separation section.

8. The system of claim 7 wherein the primary matrix retention section and the final yeast separation section are of cylindrical shape and the flow rate reduction section is of conical shape.

9. The system of claim 8 wherein the cross sectional area of the final yeast separation section is greater than the cross sectional area of the primary matrix retention section.

10. The system of claim 1 wherein the fermentor is sized and shaped to allow sufficient dwell time of the sugar supply in the packed bed region and converted ethanol in the fluidized bed region to maximize sugar conversion to ethanol.

11. A process of fermenting a sugar supply to produce alcohol liquor in a single fermentor including a packed bed region and a fluidized bed region, the process comprising the steps of:
a. introducing a yeast retention matrix and a yeast to the fermentor in the packed bed region;
b. directing the sugar supply into the packed bed region at a selected flow rate;
C. maintaining the sugar supply flow substantially fixed at the selected flow rate to permit the sugar supply to dwell in the packed bed region long enough to maximize conversion; and
d. drawing fermented alcohol liquor from the fermentor.

12. The process of claim 11 further comprising the step of introducing a yeast nutrient to the fermentor prior to the step of directing the sugar supply into the packed bed region.

13. The process of claim 11 further comprising the step of calculating fermentor dimensions to allow sufficient dwell time of the sugar supply in the packed bed region and converted ethanol in the fluidized bed region to maximize sugar conversion to ethanol prior to the step of introducing the yeast retention matrix to the fermentor.

14. A fermentor arranged to ferment a sugar supply to produce alcohol liquor, the fermentor comprising:
a. a packed bed region arranged to receive the sugar supply and to retain a yeast bed therein, wherein the sugar supply is converted into ethanol and carbon dioxide substantially in the packed bed region; and
b. a fluidized bed region for receiving the ethanol and carbon dioxide from the packed bed region, wherein the fluidized bed is arranged to permit yeast cell retention therein.

15. The fermentor of claim 14 further comprising a retention matrix and a yeast nutrient in the packed bed region, wherein the yeast nutrient is selected to aid yeast flocculation.

16. The fermentor of claim 14 including a primary matrix retention section and a final yeast separation section.

17. The fermentor of claim 14 including a flow rate reduction section between the primary matrix retention section and the final yeast separation section.

18. The fermentor of claim 17 wherein the primary matrix retention section and the final yeast separation section are of cylindrical shape and the flow rate reduction section is of conical shape.

19. The fermentor of claim 18 wherein the cross sectional area of the final yeast separation section is greater than the cross sectional area of the primary matrix retention section.

20. The fermentor of claim 14 sized and shaped to allow sufficient dwell time of the sugar supply in the packed bed region and converted ethanol in the fluidized bed region to maximize sugar conversion to ethanol.
